(19) United States
(12) Patent Application Publication
(10) Pub. No.: US 2005/0019310 A1
(43) Pub. Date: Jan. 27, 2005

(54) Method for culturing and expansion of mammalian undifferentiated epidermal kerainocytes exhibiting stem cell characteristics

(76) Inventor: Richard John Isaacs, Ottawa (CA)

Correspondence Address: J Wayne Anderson, P O Box 1266, Station B, Ottawa, Ontario, KIP 5R3, CA

(21) Appl. No.: 10/499,716

(22) PCT Filed: Dec. 20, 2002
(86) PCT No.: PCT/CA02/01980

Related U.S. Application Data
(60) Provisional application No. 60/341,866, filed on Dec. 21, 2001.

(51) Int. Cl. A61K 38/17; A61K 45/00

(52) U.S. Cl. 424/93.7; 514/12

(57) ABSTRACT

The invention disclosed relates to the culturing of mammalian e.g. human cells, and in particular to the culturing and expansion of substantially undifferentiated human epidermal keratinocytes exhibiting stem cell-like characteristics, and in co-culture with human dermal fibroblasts utilizing a low calcium serum free and animal by-product free medium derived from a commercially available medium. The medium consists of a commercially available basal medium, and a single fibroblast growth factor (FGF) or a mimic thereof e.g. FGF7/KGF. A method is also disclosed for treating mammalian e.g. human skin wound injuries, by applying to the wound an effective amount of substantially pure mammalian e.g. human epidermal stem cell-like keratinocytes in a substantially undifferentiated state, and optionally additionally, dermal fibroblasts e.g. human dermal fibroblasts. Both cell types can be grown separately or in a co-culture for application purposes.

### BACKGROUND OF THE INVENTION

This invention relates to the culturing of mammalian e.g. human cells, and in particular to the culturing and expansion of substantially undifferentiated human epidermal keratinocytes exhibiting stem cell characteristics, and in co-culture with human dermal fibroblasts utilizing a serum free and animal by-product free medium derived from a commercially available medium.

Up to this point in wound healing and other areas that utilize the growth of keratinocytes and fibroblasts or the co-culturing of both, no one has initiated the growth of human keratinocytes that exhibit stem cell like characteristics, for use in wound healing and/or for studies using a true skin model consisting of a complete dermis and epidermis.

Moreover, Keratinocyte Serum Free Medium used by others has contained animal by products such as dialyzed serum (bovine usually), or additives such as pituitary extract (bovine) or other animal derived compounds.

Two examples of commercially available Keratinocyte Serum Free Medium for primary and secondary human keratinocyte culturing are:

GIBCO® KSFM (from Invitrogen Life Technologies Burlington Ontario Canada) with the supplementation of prequalified human recombinant Epidermal Growth Factor 1-53 (EGF 1-53), and Bovine Pituitary Extract (BPE). References: Daley, J. P. , Epstein, D. A. and Hawley-Nelson, P (1990) FOCUS 12, 68, and Donovan, J. (1998) FOCUS 20,38.

GIBCO® Defined Keratinocyte-serum free medium (KSFM), which replaces the bovine pituitary extract (BPE) found in previous media with growth promoting additives including insulin, epidermal growth factor (EGF) and fibroblast growth factor (FGF7/KGF). See Culture of Human Keratinocytes in Defined Serum-Free Medium, David A. Judd et al., Focus 19 Number 1, 2001. See also published PCT application no. WO98/16629, published 23 Apr. 1998.

Some simple defined cell growth media, sometimes called “basal” media, comprise a number of ingredients basic to cell viability, including amino acids, vitamins, organic and inorganic salts and sugars, each ingredient being present in an amount which supports the viability of mammalian epithelial cells in vitro, in a solution of double de-ionized distilled water.

One example of such a “basal” medium, which is both serum free and animal by-products free, for culturing primary and secondary human epidermal keratinocytes is the GIBCO® keratinocyte serum free medium (KSFM), catalogue no. 17005-042 (available from Invitrogen Life Technologies, Burlington, Canada. This is a low calcium concentration medium, which does not include any cell growth additives.

The following references, the disclosures of which are incorporated herein by reference, also describe the GIBCO (now Invitrogen) serum-free keratinocyte growth media.

Maciag, T., Newmore, R. E., Weinstein, R., and Gilchrest, B. A. An Endocrine Approach to the Control of Epidermal Growth: Serum-Free Cultivation of Human Keratinocytes Science 211, 1452-1454 (1981)

Tsao, M. C., Walthall, B. J., and Ham, R. G. Colonal Growth of Normal Human Epidermal Keratinocytes in a Defined Medium, J. Cell. Physiol. 110, 219-229 (1982)

Boyce, S. T., and Han, R. G. Calcium Regulated Differentiation of Normal Epidermal Keratinocytes in Chemically Defined Colonal and Serum Free Serial Culture. J. Invest. Dermatol 81, 33S44S (1983)

Pittelkow, M. R., and Scott, R. E. New Techniques for the In Vitro Culture of Human Skin Keratinocytes and Perspectives on their use for Grafting of Patients with Excessive Burns Mayo Clinic Proceedings 61, 771-777 (1986)

Pirisi, L., Yasumoto, S., Feller, M., Doniger, J., and DiPaolo, J. A. Transformation of Human Fibroblasts and Keratinocytes with Human Papillomavirus Type 16 DNA Carcinogenesis. 9, 1573-1579 (1988)

Pirisi, L. Yasumoto, S., Feller, M., Doniger, J. and DiPaolo, J. A. Transformation of Human Fibroblasts and Keratinocytes with Papillomavirus Type 16 DNA J. Virol. 61, 1061-1066 (1987)

Lechner, J. F. and LaVeck M. A. A Serum-Free Method for Cultivating Normal Human Bronchial Epithelial Cells at Clonal Density J. Tiss. Cult. Meth 9, 43-48 (1985)

Further, the co-culturing of the human dermal fibroblasts and human epidermal keratinocytes has proven very difficult up to now because of the different calcium requirements of the two phenotypes for survival.

There has been nothing produced thus far by companies in the wound healing business or in laboratories working with keratinocytes indicating the isolation and growth of solely stem cell-like epidermal keratinocytes, or a co-culture of both human dermal fibroblasts and human epidermal keratinocytes originating from either epidermal stem cells or epidermal progenitors using a serum free, animal by product free medium. Also, the in vitro growth of massive numbers of stem like keratinocytes and their maintenance in a substantially undifferentiated state with the intent to add to a wound site has not been accomplished.

At present, the ideal cells to use for population expansion are those obtained from neonatal foreskin. However, this requires a reliable source of supply, and intense screening for human infections. Further, if cells from the patient were to be used there would be a time delay in expanding those cells for transplantation.

### SUMMARY OF THE INVENTION

According to one aspect of the invention, a method is provided for selective culturing and expansion of substantially pure mammalian e.g. human epidermal keratinocytes, exhibiting stem cell-like characteristics in a substantially undifferentiated state, comprising

(a) providing a culture of primary, cryo-preserved or actively growing mammalian epidermal keratinocytes in vitro, and

(b) culturing the keratinocytes in a cell culture medium consisting of a serum free and animal by-products free basal medium capable of supporting the growth of mammalian epithelial cells in vitro and a fibroblast growth factor (FGF) or a mimic thereof, wherein the medium is capable of culturing and expansion of substantially pure mammalian e.g. human epidermal keratinocytes exhibiting stem cell-like characteristics in a substantially undifferentiated state in vitro.

Examples of a keratinocyte starting material include isolated stem cell keratinocytes from an allogenic or autologous source using the proven Jones and Watt, “Separation of Human Epidermal Stem Cells from Transit Amplifying Cells on the Basis of Differences in Integrin Function and Expression” Cell 73:713-723 (1993), the disclosure of which is incorporated herein by reference. Differentiated keratinocytes either stored in liquid nitrogen or actively growing in vitro can also be used simply by their adaptation to the novel medium according to the invention.

According to another aspect of the invention, a method is provided for co-culturing of substantially pure mammalian e.g. human epidermal stem cell-like keratinocytes in a substantially undifferentiated state, and human dermal fibroblasts, comprising

(a) providing mammalian epidermal keratinocytes and mammalian dermal fibroblasts, either from primary tissue or from in vitro cultured stocks of growing or cryo-preserved cells, and.

(b) co-culturing the keratinocytes and fibroblasts in a cell culture medium consisting of a serum free and animal by-products free basal medium capable of supporting the growth of mammalian epithelial cells in vitro and a fibroblast growth factor (FGF) or a mimic thereof, wherein the medium is capable of culturing and expansion of substantially pure mammalian e.g. human epidermal keratinocytes exhibiting stem cell-like characteristics in a substantially undifferentiated state, while maintaining dermal fibroblast viability, in vitro.

According to yet another aspect of the invention, a novel cell culture medium is provided, consisting of a serum free and animal by-products free basal medium capable of supporting the growth of mammalian epithelial cells in vitro and a fibroblast growth factor (FGF) or a mimic thereof, wherein the medium is capable of culturing and expansion of substantially pure mammalian e.g. human epidermal keratinocytes exhibiting stem cell-like characteristics in a substantially undifferentiated state in vitro.

It will be appreciated that this novel medium will support the growth of epidermal stem cell-like keratinocytes in a substantially undifferentiated state and also support the growth of dermal fibroblasts in co-culture with the keratinocytes.

In one embodiment of this aspect of the invention, the FGF is FGF7/KGF. A source of FGF7/KGF is human recombinant protein expressed in E. coli, (Upstate Cell Signalling Solutions, catalogue no. 01-118.

It will be appreciated by those skilled in the art that other FGFs and mimics may also be used as the growth supplement according to the invention ie. provided that they are ones which induce keratinocyte proliferation, rather than differentiation e.g. FGF-10. According to some recent publications, stem cell-like keratinocytes express only, and are the only phenotype to express the KGF receptor and another yet undefined one that binds FGF-10. See Werner S., 1998, Keratinocyte Growth factor, a unique player in epithelial repair processes, Cytokine and Growth Factor Review, 9, 153-165, and Braun S. et al., 2002, Growth factors in development, repair and disease, Futura, 17, 61-70.

A “mimic” is defined herein as a ligand, which mimics the biological properties/characteristics of, in this case, such an FGF.

In another embodiment, the FGF7/KGF is included in an amount of 5-10 ng/ml of medium.

In yet another embodiment, the amount of FGF7/KGF is 10 ng/ml of medium.

In still another embodiment of this aspect of the invention, the medium comprises, as an example of the basal medium, the prior GIBCO® Keratinocyte Serum Free Medium (KSFM) catalogue no. 17005-042 described above, and instead of the growth factors included in the medium ie. Epidermal Growth Factor (EGF) and Bovine Pituitary Extract (BPE), a single fibroblast growth factor (FGF) e.g. FGF7/KGF is added in an amount of 5-10 ng/ml of medium. Note that there is no indication in any of the literature that this GIBCO® KSFM catalogue no. 17005-042 contains any ingredients including compounds of animal origin.

It will be appreciated by those skilled in the art that other similar low calcium ion concentration commercial basal media can be used, such as Dulbecco's Modified Eagle's Medium/Ca⁺⁺ supplement-free Ham's Nutrient Mixture F-12 in a 1:1 mixture (product no. D6434).

The serum free and animal by-products free basal medium of the invention, comprises various ingredients basic to cell viability, wherein each ingredient is included in an amount which supports the viability of mammalian epithelial cells in vitro, in a solution of de-ionized distilled water.

Ingredients which may be included are amino acids, vitamins, organic and inorganic salts and sugars.

Specific amino acid ingredients include L-alanine, L-arginine, L-aspargine, L-aspartic acid, L-cysteine, L-glutamic acid, L-glutamine, glycine, L-histidine, L-isoleucine, L-leucine, L-lysine, L-methionine, L-phenylanaline, L-proline, L-serine, L-threonine, L-tryptophan, L-tyrosine, and L-valine.

Vitamin ingredients include biotin, choline chloride, D-Ca++-pantothenate, folic acid, I-inositol, niacinamide, pyridoxine, riboflavin, thiamine and Vitamin B12.

Inorganic salts include CuSO₄, FeSO₄, KCl, a magnesium salt(e.g. MgCl₂), a manganese salt (e.g. MnCl₂), sodium acetate, NaCl, NaHCO₃, Na₂HPO₄, Na₂SO₄, and ions of the trace elements selenium, silicon, molybdenum, vanadium, nickel, tin and zinc. These trace elements may be provided in various forms, such as salts.

These ingredients may be obtained commercially, for example, from Sigma of St. Louis, Mo.

According to yet another aspect of the invention, a method is provided for treating mammalian e.g. human skin wound injuries, by applying to the wound an effective amount of substantially pure mammalian e.g. human epidermal stem cell-like keratinocytes in a substantially undifferentiated state exhibiting MHC (HLA) pro expressers, and optionally additionally dermal fibroblasts e.g. human dermal fibroblasts. Both cell types can be grown separately or in a co-culture for application purposes.

According to one embodiment of this aspect of the invention, we have found it advantageous to start the wound healing process as soon as possible with the addition of pre-screened allogenic human dermal fibroblasts and previously grown and stored stem cell-like epidermal keratinocytes exhibiting LDN as down regulated MHC molecules. Concurrently, the patients cells are harvested and expanded in the novel medium to ensure a homogeneous population of stem cell-like epidermal keratinocytes and dermal fibroblasts, to be subsequently applied to the wound site in order to promote optimal healing.

In either case, prior to treatment, the epidermal stem cell-like keratinocytes and dermal fibroblasts, either individually or in a co-culture, may be first transferred to a suitable matrix or carrier e.g. biodegradable micro spheres or scaffolds, which may be coated with a material which has a higher affinity for cellular adhesion, e.g. collagen. A system of this nature is described in U.S. Pat. No. 5,980,888, the disclosure of which is incorporated herein by reference.

Individually the dermal fibroblasts are added first to the matrix and grown in their respective media, and when at 70 to 80 percent confluent the stem cell-like keratinocytes grown in the novel medium according to the invention e.g. PG Defined KSFM, are added to the pre-washed matrix containing the fibroblasts and fresh novel medium e.g. PG Defined KSFM is added as the growth medium for both cell types.

Controlled differentiation of the stem keratinocytes grown in the novel medium by the addition of calcium or keratinocytes grown on a matrix that will slowly release calcium for keratinocyte differentiation. The slow release matrix can also be used for the co-culture growth of the stem keratinocytes and dermal fibroblasts.

From an existing co-culture both types of cells can be added simultaneously to a matrix. This has been done successfully using Cytodex® micro spheres coated with 10 μg/ml collagen Type IV from Sigma

Co-cultures of human dermal fibroblasts and substantially undifferentiated human epidermal stem cell-like keratinocytes have been maintained invitro for 23 days undergoing 2 subcultures but still retaining their original characteristics.

To the inventor's knowledge no other medium up to now has been developed to both provide for the expansion of the stem cell-like keratinocytes in a substantially undifferentiated state, but still retaining differentiation potential, while at the same time sustaining dermal fibroblast viability. Yet in co-culture both cell types retain the capability of expansion in the novel medium.

Another major advantage of the novel medium is that due to the absence of animal by-products, cross transmittance of disease is avoided.

The stem keratinocytes and dermal fibroblasts are provided from either autologous or allogenic supplies of keratinocytes and fibroblasts.

The stem cell-like epidermal keratinocytes and dermal fibroblasts could also be added directly to the wound as a viable mixed culture of stem cell-like keratinocytes and dermal fibroblasts, the stem cell-like keratinocytes exhibiting increasing HLA quantities along with the fibroblasts ensures an accelerated and more complete epithelization of the wound site preventing a miss matched tolerance.

### BRIEF DESCRIPTION OF THE DRAWING

FIG. 1 includes micro-photographs illustrating normal growth of keratinocytes in a commercially available KSFM ie the basal GIBCO® medium catalogue no. 17-005-042, and the novel PG Defined KSFM according to the invention.

FIG. 2 illustrates a comparison of cultures grown in commercially available KSFM ie. GIBCO® (KSFM), supplemented with growth promoting additives including EGF and BPE, and cultures grown in the novel PG Defined KSFM according to the invention, including 10 ng/ml of medium of FGF7/KGF.

FIG. 3 includes Western blots showing the 40 and 50 kDa proteins that represent cytokeratins 14 and 19, the marker cytokeratins for stem keratinocyte identification. The western blots also show the results of the addition of 10% fetal bovine serum to the growing keratinocytes for a 24 hour period.

FIG. 4 includes micro-photographs of human epidermal Keratinocytes grown in the PG Defined KSFM including 10 ng/ml of FGF7/KGF, and added to a culture of human dermal fibroblasts grown for 5 days as a co-culture in the PG Defined medium including 10 ng/ml of FGF7/KGF, then subcultured

### DETAILED DESCRIPTION OF THE INVENTION

### Culture of Undifferentiated Stem Like Keratinocytes

The culturing of isolated stem keratinocytes using the Jones-Watts⁴ isolation procedure, the disclosure of which is incorporated herein by reference, for selective growth of a normal keratinocyte population, incorporating the use of a novel medium, which targets the growth and maintenance in an undifferentiated stage of stem like keratinocytes is described herein.

### Example of a Typical Cell Culture Method

Cells taken directly from a neo-natal foreskin sample (as an example source of supply) enabled the procurement of epidermal cells, and eliminating any chance of cross-contamination, or organ rejection if donor cells had been used. The skin is incubated in a sterile dispase solution enabling the epidermis to be peeled away from the dermis with sterile forceps. The epidermis was then disaggregated into a single cell suspension with 0.05% trypsin and 0.016% EDTA, for 20-30 minutes at 37° C., while stirring gently. When a single cell suspension was achieved, the cells were centrifuged at 1000 rpm approximately 160 times gravity for 5 min, the trypsin/EDTA supernatant removed, and a volume of fresh inoculum medium added to disperse the cells. The cells were then brought up into a syringe containing collagen coated micro spheres and rotated very slowly to enable the stem cells which have a higher affinity for collagen to adhere to the surface of the micro spheres in a short time i.e. About 5 minutes. The remainder of the unattached cells, mainly cells in more advanced stages of differentiation were removed at this time. The micro spheres at this point are ready to apply to the wound site.

This approach selectively selects the keratinocytes with stem cell-like characteristics, with applications that can be utilized in various domains including epidermal maintenance and repair.

Work on developing a serum-free and animal by-products free medium for stem cell-like keratinocytes, by, substantially eliminating the keratinocytes from differentiating, is also described herein.

It is known that stem keratinocytes comprise only approximately 20% of the total keratinocyte population collected from human epidermal skin sections. The novel medium according to the invention amplifies only the keratinocytes exhibiting stem cell-like characteristics and does not promote the differentiation of these keratinocytes, therefore substantially eliminating differentiated cells resulting in a pure culture of keratinocytes with stem cell-like characteristics.

Probing of these cells by cytofluorimetry using cyto-keratin probes to check more precisely for stem cell-like keratinocytes, indicates that the cells cultured and grown in the novel medium according to the invention, show a positive indication that over 90% of the viable cells exhibit the keratin expression pattern of stem keratinocytes after growth in the novel medium for 5 days.

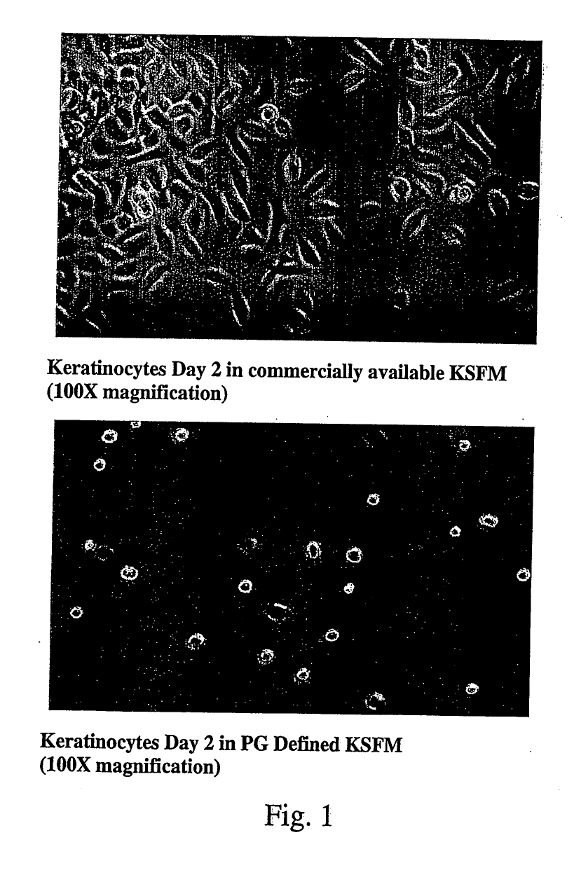

FIG. 1 includes micro-photographs illustrating normal growth of keratinocytes in commercially available KSFM ie the basal GIBCO® medium catalogue no. 17-005-042, and the novel PG Defined KSFM according to the invention. It is to be noted that in the first few days of growth the cell in PG Defined KSFM are rounded, smaller but have attached to a growth surface, whereas the cells in the commercially available KSFM are spreading out and starting to proliferate. The PG Defined KSFM cells have started to spread out on Day 2 and will continually grow in colonies thereafter.

### EXAMPLE 1

Experiments adding various amounts of FGF7/KGF to the basal GIBCO® KSFM catalogue no. 17005-042, were completed to indicate the optimal concentration of KGF addition to the basal medium. The amounts added were 10, 5, 2.5 and 1 ng/ml of medium. After an 8 day period, the 10 ng/ml addition provided superior growth of the stem cell-like keratinocytes. The 5 ng/ml version showed good cell viability, but slower stem cell-like keratinocyte growth. The lower concentrations showed inadequate stem cell-like keratinocyte growth. We did not go above 10 ng/ml, because of the high cost of the compound. However, at this point, the graph is levelling off in any case.

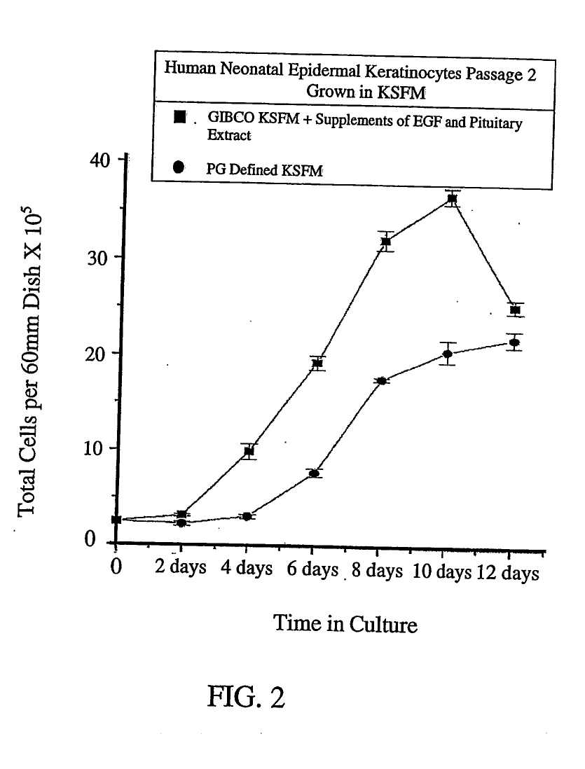

FIG. 2 is a graph illustrating the comparison of cultures grown in commercially available KSFM ie GIBCO® KSFM, supplemented with growth promoting additives including EGF and BPE, and cultures grown in the novel PG Defined KSFM according to the invention, including 10 ng/ml of medium of FGF7/KGF. The growth curve shows that there is a definite lag period of the keratinocytes grown in the PG Defined KSFM when compared to the commercial KSFM.

A novel feature of this invention is that it is not just growing keratinocytes for wound healing applications, but selecting and amplifying the growth of strongly binding beta 1 integrin stem cell-like keratinocytes, exhibiting the 5-14 and 19 keratin expression characteristic of basal cells. Either by the isolation of epidermal stem cell-like keratinocytes using the Jones, Watt's isolation method, or the growing of epidermal stem cell-like keratinocytes in the novel medium according to the invention e.g. the PG Defined KSFM, increases the number of beta 1 integrin dominant keratinocytes

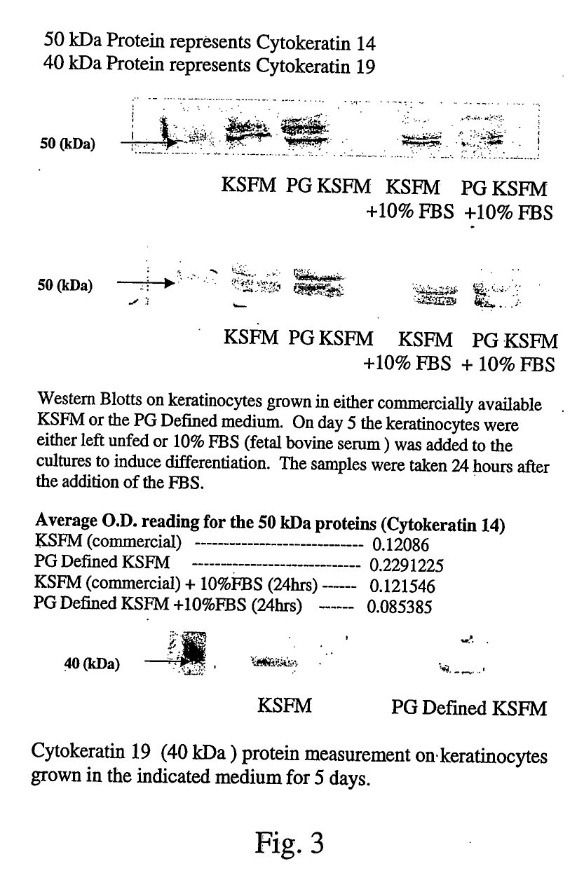

FIG. 3 includes Western blots showing the 40 and 50 kDa proteins that represent cytokeratins 14 and 19, the marker cytokeratins for stem keratinocyte identification. The western blots also show the results of the addition of 10% fetal bovine serum to the growing keratinocytes for a 24 hour period. The keratinocytes grown in the PG Defined KSFM show that keratin 14 expression (50 kDa protein) is greatly reduced indicating the cells have started to differentiate.

### EXAMPLE 2

Allogenic stem keratinocytes have been isolated utilizing a small number of cells, using methods described by Jones et al. 1995², the disclosure of which is incorporated herein by reference. After isolation of a small number of the stem keratinocytes, the cells are grown in the PG Defined KSFM including 10 ng/ml of medium of FGF7/KGF, into a homogeneous culture of keratinocytes with stem cell-like characteristics, therefore reducing the risk of immunogenicity. 90+% of the viable cells were found to exhibit keratin 5-14 expression pattern of keratinocytes progenitors.

Autologous keratinocytes were expanded in the novel PG Defined KSFM on either a commercially constructed imatrix or in a culture, giving a population of keratinocytes with stem cell like characteristics and then transferred to the wound site using a variety of methods of application.

This method, using the novel medium (PG Defined KSFM) amplifies small numbers of either allogenic or autologous keratinocytes to vast numbers of stem cell-like keratinocytes for wound healing, therefore greatly increasing the number of non differentiated keratinocytes added to the wound site.

This is further illustrated by using the PG Defined KSFM on in vitro cultured differentiated keratinocytes (grown in a commercial KSFM). The keratinocytes exposed to the PG Defined KSFM for a short period exhibit an increased number of cells expressing the beta 1 integrin and there affinity for collagen therefore surviving and proliferating as a virtually homologous population of keratinocytes exhibiting stem cell characteristics. This is indicated below.

### EXAMPLE 3

Experimental results obtained by (Jones and Watts⁴), the disclosure of which is incorporated herein by reference, indicate that the total number of stem keratinocytes in a primary epidermal cell population is approximately 20%. Their method for the isolation of stem keratinocytes is a timed adherence of the cells to a 10% collagen coating using 5 minutes as a maximum adherence time. When adhesion tests were performed on keratinocytes grown in the GIBCO KSFM+EGF+Pituitary Extract, only 2-5% of the keratinocytes complied whereas 60-65% of the keratinocytes appeared on the same collagen base after 5 minutes when grown in PG Defined KSFM, including 10 ng/ml of FGF7/KGF.

When keratinocytes were grown in the commercially available GIBCO® KSFM+supplements (Epidermal Growth Factor and Pituitary Extract) and then transferred after sub-culturing to either their original growth medium GIBCO KSFM+supplements or PG Defined KSFM including 10 ng/ml of FGF7/KGF, the results were as follows when subjected to the adhesion test for stem keratinocytes developed by Jones and Watts. Type IV Collagen (Sigma) at a concentration of 100 μg/ml coated plastic culture dishes were used

| Time in the | | |
|---|---|---|
| respective medium | Medium Type | % Adhesion |
| 5 minutes | PG Defined KSFM (10 ng/ml KGF) | 42.4% |
| | GIBCO KSFM + Supplements | less than 1% |
| 15 minutes | PG Defined KSFM (10 ng/ml KGF) | 47% |
| | GIBCO KSFM + Supplements | 6.3% |

Moreover, keratinocytes that quickly adhere to a collagen base are shown by the Jones and Watt⁴ procedure to possess the stem cell characteristic collagen binding integrins, the same integrins that appear in cells grown in the novel medium.

By way of comparison, the conventional medium for keratinocytes is either commercially available KSFM with a variety of different supplements depending on the manufacturer, or a DMEM medium with a supplement of dialyzed fetal bovine serum at a concentration of 10%. Keratinocytes grown in such conventional media grow in monolayers, whereas cells grown in the novel medium (PG Defined KSFM) grow in colonies, and appear to be smaller and exhibit characteristics of stem keratinocytes as described in the literature. See FIG. 1.

### Co-Culture of Epidermal Stem Cell-Like Keratinocytes and Dermal Fibroblasts in the Novel Medium (PG Defined KSFM) According to the Invention

The test results were positive in the experiments performed. It was noted that dermal fibroblast viability remained at about 100%, and putative stem keratinocytes remain viable without feeding for at least 14 days. This results in a valuable tool when using both types of allogenic cells for application to wound healing. The allogenic dermal fibroblasts and stem cell-like keratinocytes can be cultured on the same micro spheres or on separate micro spheres, and stored in the same package, providing an animal by product-free environment. This means that a packaged product for complete epithelization of a wound area, can be produced, packaged, stored and shipped when required.

The mechanism involves the dermal fibroblasts sinking to the base of the wound, with the stem keratinocytes forming an epidermal layer on top. By using the stem keratinocytes in large numbers, once applied to the wound, they start differentiating by using the recipient's own calcium. Similarly, the dermal fibroblasts also access the bodies' natural calcium to enhance their proliferation. Accordingly, this combined system of epidermal stem cell-like characteristic human keratinocytes and human dermal fibroblasts when added to a wound site in the novel medium (PG Define KSFM) could provide a complete package for wound healing.

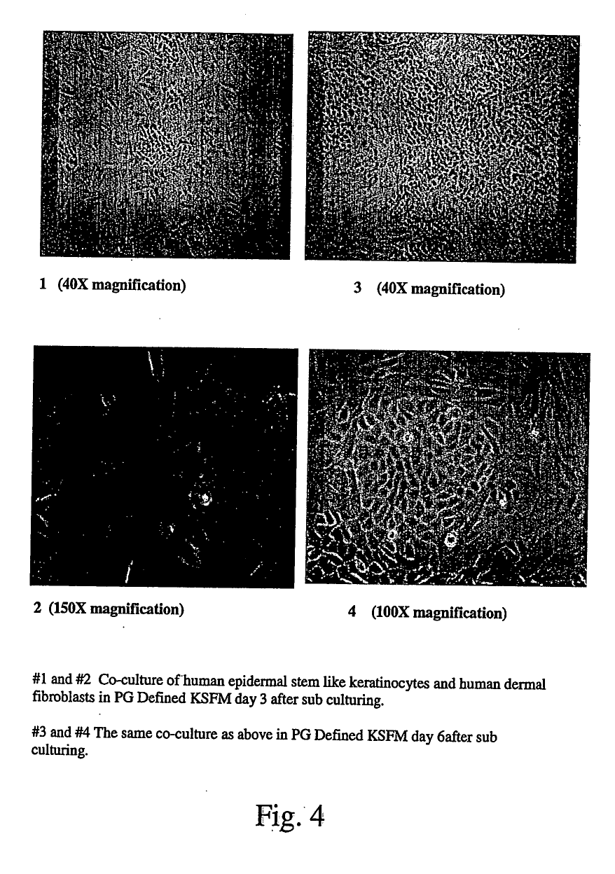

FIG. 4 includes micro-photographs of human epidermal Keratinocytes grown in the PG Defined KSFM including 10 ng/ml of FGF7/KGF, and added to a culture of human dermal fibroblasts grown for 5 days as a co-culture in the PG Defined medium including 10 ng/ml of FGF7/KGF, then sub-cultured. Illustrated is the growth of the co-culture after sub- culturing. Colony formation and the growth of these colonies is also illustrated.

I have further observed that in a co-culture the epidermal stem cell-like keratinocytes align themselves against the dermal fibroblasts producing what appears to be a collagen layer separating them, indicating the keratinocytes use the fibroblasts as a feeder layer. This duplicates the natural formation of whole skin where as the keratinocytes sit upon the fibroblasts separated by a collagen layer to produce a viable dermal and epidermal whole skin formation.

The procedure for cloning, generating and maintaining a substantially pure culture of hundreds of thousands of substantially undifferentiated epidermal stem cell-like keratinocytes that would reduce wound healing time is novel. The specific serum free animal by product free medium for the growth of epidermal stem cell-like keratinocytes and it's ability to sustain dermal fibroblast viability in pure culture and dermal fibroblast growth in co-culture with keratinocytes is also novel. This method can use either autologous or allogenic skin cells for the purpose of transplanting. Stem cell-like keratinocytes have been isolated in the past from mixed populations of primary actively differentiating keratinocytes at various stages, that continue to differentiate when cultured in vitro. This procedure eliminates this. A major advantage of the method according to the invention is to produce unlimited homogenous stocks of substantially undifferentiated epidermal stem cell-like keratinocytes for immediate use in wound healing. The ability to expand to vast numbers of substantially undifferentiated keratinocytes increases the patients chances of complete wound epithelization quickly by using allogenic cultured cells initially and then the addition of the patients' own cells which have been expanded to promote complete wound closure. Acticel, a non autologous version of Epicel (manufactured by BioSurface Technology BST) is currently being used as a carrier in clinical trials to add keratinocytes to the wound bed.

I also contemplate a complete new approach using human substantially undifferentiated keratinocytes with stem cell-like characteristics, in conjunction with human dermal fibroblasts, grown in an animal by- product free environment, as a 3D Cell Model or 2D cell Model for the testing of drugs, toxic gases, cosmetics and any other product topically applied that enters (or not) the body via the skin. The 3D and 2D Cell Models can be expanded as a co-culture utilizing keratinocytes and fibroblasts grown in the novel medium or supported by a biodegradable matrix enabling total cell removal from the preservation, transportation or testing environment.

### References

1. U.S. Pat. No. 5,980,888 Keratinocytes Attached to Micro carriers for Treatment of skin wounds.

Dimoudis; Nikolaos, Hartinger, Anton

Issued Nov. 9, 1999/Apr. 23, 1997

Applicant Roche Diagnostics Gmbh, Mannheim Germany

2. Stem Cell Patterning and Fate in Human Epidermis

Philip H. Jones, Steven Harper, and Fiona Watt

Cell Vol. 80, 83-93, Jan. 13, 1995

3. Epidermal Stem Cells as Targets for Gene Transfer

Fiona M. Watt

Human Gene Therapy 11:2261-2266 (Nov. 1, 2000)

4. Jones and Watt, “Separation of Human Epidermal Stem Cells from Transit Amplifying Cells on the Basis of Differences in Integrin Function and Expression” Cell 73:713-723 (1993).

1. A method for selective culturing and expansion of substantially pure mammalian e.g. human epidermal keratinocytes, exhibiting stem cell-like characteristics in a substantially undifferentiated state, comprising
(a) providing a culture of primary, cryo-preserved or actively growing mammalian epidermal keratinocytes in vitro, and
(b) culturing the keratinocytes in a cell culture medium consisting of a serum free and animal by-products free basal medium capable of supporting the growth of mammalian epithelial cells in vitro and a fibroblast growth factor (FGF) or a mimic thereof, wherein the medium is capable of culturing and expansion of substantially pure mammalian e.g. human epidermal keratinocytes exhibiting stem cell-like characteristics in a substantially undifferentiated state in vitro.

2. A method according to claim 1, wherein the FGF is FGF7/KGF.

3. A method according to claim 2, wherein the FGF7/KGF is included in an amount of 5-10 ng/ml of medium.

4. A method according to claim 3, wherein the amount of FGF7/KGF is 10 ng/ml of medium.

5. A method according to claim 1, wherein the keratinocyte starting material comprises isolated stem cell keratinocytes from an allogenic or autologous source.

6. A method for co-culturing of substantially pure mammalian e.g. human epidermal stem cell-like keratinocytes in a substantially undifferentiated state, and human dermal fibroblasts, comprising
(a) providing mammalian epidermal keratinocytes and mammalian dermal fibroblasts, either from primary tissue or from in vitro cultured stocks of growing or cryo-preserved cells, and.
(b) co-culturing the keratinocytes and fibroblasts in a cell culture medium consisting of a serum free and animal by-products free basal medium capable of supporting the growth of mammalian epithelial cells in vitro and a fibroblast growth factor (FGF) or a mimic thereof, wherein the medium is capable of culturing and expansion of substantially pure mammalian e.g. human epidermal keratinocytes exhibiting stem cell-like characteristics in a substantially undifferentiated state, while maintaining dermal fibroblast viability, in vitro.

7. A method according to claim 6, wherein the FGF is FGF7/KGF.

8. A method according to claim 7, wherein the FGF7/KGF is included in an amount of 5-10 ng/ml of medium.

9. A method according to claim 8, wherein the amount of FGF7/KGF is 10 ng/ml of medium.

10. A method according to claim 6, wherein the keratinocyte starting material comprises stem cell keratinocytes isolated from an allogenic or autologous source, and wherein the dermal fibroblast starting material comprises dermal fibroblasts isolated from an allogenic or autologous source.

11. A cell culture medium, consisting of a serum free and animal by-products free basal medium capable of supporting the growth of mammalian epithelial cells in vitro and a fibroblast growth factor (FGF) or a mimic thereof, wherein the medium is capable of culturing and expansion of substantially pure mammalian e.g. human epidermal keratinocytes exhibiting stem cell-like characteristics in a substantially undifferentiated state in vitro.

12. A medium according to claim 11, wherein the FGF is FGF7/KGF.

13. A medium according to claim 12, wherein the FGF7/KGF is included in an amount of 5-10 ng/ml of medium.

14. A medium according to claim 13, wherein the amount of FGF7/KGF is 10 ng/ml of medium.

15. A method for treating mammalian e.g. human skin wound injuries, by applying to the wound an effective amount of substantially pure mammalian e.g. human epidermal stem cell-like keratinocytes in a substantially undifferentiated state exhibiting MHC (HLA) pro expressers, and optionally additionally mammalian dermal fibroblasts eg. human dermal fibroblasts.

16. A method according to claim 15, wherein both cell types are grown separately or in a co-culture for application purposes.

17. A method according to claim 15, wherein the keratinocytes are previously grown and stored stem cell-like human epidermal keratinocytes exhibiting LDN as down regulated MHC molecules, and wherein the dermal fibroblasts are pre-screened allogenic human dermal fibroblasts.

18. A method according to claim 17, wherein concurrently, the patients' cells are harvested and expanded in the novel medium as defined in any one of claims 11-14, to provide a homogeneous population of stem cell-like epidermal keratinocytes and dermal fibroblasts, and subsequently applied to the wound site in order to promote optimal healing.

19. A method according to claim 15, prior to treatment, the epidermal stem cell-like keratinocytes and dermal fibroblasts, either individually or in a co-culture, may be first transferred to a suitable matrix or carrier e.g. biodegradable micro spheres or scaffolds, which may be coated with a material which has a higher affinity for cellular adhesion, e.g. collagen.

20. A method according to claim 15, wherein the stem keratinocytes and dermal fibroblasts are provided from either autologous or allogenic supplies of keratinocytes and fibroblasts.
